SEQUENCE LISTING (49 sequences)

SEQ ID NO: 1 - DNA, 234 bp, SARS E gene
atggcatactcttttgtgtctgaggaaactggcactctgatcgtgaactctgtactgctgtttctcgcttttgtggtattcctgctggtcactctcgctatcctcactgctcttcgtctgtgtgcctactgttgtaatatcgtgaacgtgtctctggttaagcctactgtgtatgtgtattctcgtgtgaaaaatctcaattcttctgaaggagttcccgatctgctggtctag

SEQ ID NO: 2 - DNA, 666 bp, SARS M gene
atggcagataacggcactattactgtggaggaactgaaacaactgctggaacaatggaacctcgtaatcggctttctctttctggcttggattatgttgttacagtttgcgtattctaatcgtaaccgtttcctctacattattaagctcgttttcctgtggttgttgtggcctgtaactcttgcttgctttgtgcttgctgctgtctatcgtatcaactgggttactggtggtattgctatcgctatggcttgtattgtaggcttgatgtggctgtcttatttcgttgcttctttccgtctgtttgctcgtactcgctctatgtggtcctttaatcctgagactaatatcctgctgaatgttccgctccgtggtactatcgttactagaccgctgatggaatctgaactggttattggtgccgtcattatccgtggtcatttgcgtatggctggtcactctctgggtcgttgcgatattaaggatctgccaaaggaaatcactgtagccacttctcgtactctgtcttactataaactcggtgcatcgcaacgtgtgggaactgattcgggcttcgctgcgtataatcgttatcgtattggcaactataaactgaacaccgaccacgcaggctctaatgacaacatcgctctcctcgttcagtga

SEQ ID NO: 3 - DNA, 327 bp, SpikeCT gene
gatatctccggaatcaatgcgagcgtagtgaacatccagaaagagattgaccgtttgaatgaagttgctaaaaatctgaatgaacctctgattgacctccaagaactcggcaaatatgagcaatacattaaatggccttggtatgtctggttgggtttcatcgcaggtctcatcgctatcgttatggtgactattctgctgtgttgtatgacttcttgttgctcttgtctgaaaggtgcgtgttcttgtggttcttgttgtaagtttgatgaggatgattctgagccagttctgaagggtgtgaagctgcattatacctagttcgaa

SEQ ID NO: 4 - DNA, 34 bp, Artificial
  E1L primer
accatggcatactcttttgtgtctgaggaaactg

SEQ ID NO: 5 - DNA, 51 bp, Artificial
  E2L primer
acagcagtacagagttcacgatcagagtgccagtttcctcagacacaaaag

SEQ ID NO: 6 - DNA, 51 bp, Artificial
  E3L primer
tgaccagcaggaataccacaaaagcgagaaacagcagtacagagttcacga

SEQ ID NO: 7 - DNA, 54 bp, Artificial
  E4L primer
gcacacagacgaagagcagtgaggatagcgagagtgaccagcaggaataccaca

SEQ ID NO: 8 - DNA, 52 bp, Artificial
  E5L primer
accagagacacgttcacgatattacaacagtaggcacacagacgaagagcag

SEQ ID NO: 9 - DNA, 58 bp, Artificial
  E6L primer
gatttttcacacgagaatacacatacacagtaggcttaaccagagacacgttcacgat

SEQ ID NO: 10 - DNA, 62 bp, Artificial
  E7L primer
tctagaccagcagatcgggaactccttcagaagaattgagatttttcacacgagaatacaca

SEQ ID NO: 11 - DNA, 20 bp, Artificial
  E8L primer
tctagaccagcagatcggga

SEQ ID NO: 12 - DNA, 22 bp, Artificial
  M1-1U primer
tgatcatggcagataacggcac

SEQ ID NO: 13 - DNA, 37 bp, Artificial
  M1U primer
tgatcatggcagataacggcactattactgtggagga

SEQ ID NO: 14 - DNA, 49 bp, Artificial
  M1L primer
gttccattgttccagcagttgtttcagttcctccacagtaatagtgccg

SEQ ID NO: 15 - DNA, 49 bp, Artificial
  M2L primer
aagccagaaagagaaagccgattacgaggttccattgttccagcagttg

SEQ ID NO: 16 - DNA, 50 bp, Artificial
  M3L primer
agaatacgcaaactgtaacaacataatccaagccagaaagagaaagccga

SEQ ID NO: 17 - DNA, 57 bp, Artificial
  N4L primer
cgagcttaataatgtagaggaaacggttacgattagaatacgcaaactgtaacaaca

SEQ ID NO: 18 - DNA, 56 bp, Artificial
  M5L primer
caagagttacaggccacaacaaccacaggaaaacgagcttaataatgtagaggaaa

SEQ ID NO: 19 - DNA, 55 bp, Artificial
  M6L primer
ttgatacgatagacagcagcaagcacaaagcaagcaagagttacaggccacaaca

SEQ ID NO: 20 - DNA, 58 bp, Artificial
  M7L primer
caagccatagcgatagcaataccaccagtaacccagttgatacgatagacagcagcaa

SEQ ID NO: 21 - DNA, 56 bp, Artificial
  M8L primer
aacgaaataagacagccacatcaagcctacaatacaagccatagcgatagcaatac

SEQ ID NO: 22 - DNA, 59 bp, Artificial
  M9L primer
acatagagcgagtacgagcaaacagacggaaagaagcaacgaaataagacagccacatc

SEQ ID NO: 23 - DNA, 20 bp, Artificial
  M10U primer
tcctgctgaatgttccgctc

SEQ ID NO: 24 - DNA, 65 bp, Artificial
  M10L primer
gagcggaacattcagcaggatattagtctcaggattaaaggaccacatagagcgagtacgagcaa

SEQ ID NO: 25 - DNA, 50 bp, Artificial
  M11L primer
catcagcggtctagtaacgatagtaccacggagcggaacattcagcagga

SEQ ID NO: 26 - DNA, 60 bp, Artificial
  M12L primer
accacggataatgacggcaccaataaccagttcagattccatcagcggtctagtaacgat

SEQ ID NO: 27 - DNA, 31 bp, Artificial
  M13L primer
atacgcaaatgaccacggataatgacggcac

SEQ ID NO: 28 - DNA, 45 bp, Artificial
  M14L primer
aacgacccagagagtgaccagccatacgcaaatgaccacggataa

SEQ ID NO: 29 - DNA, 51 bp, Artificial
  M15L primer
tacagtgatttcctttggcagatccttaatatcgcaacgacccagagagtg

SEQ ID NO: 30 - DNA, 57 bp, Artificial
  M16L primer
ccgagtttatagtaagacagagtacgagaagtggctacagtgatttcctttggcaga

SEQ ID NO: 31 - DNA, 58 bp, Artificial
  M17L primer
cgaagcccgaatcagttcccacacgttgcgatgcaccgagtttatagtaagacagagt

SEQ ID NO: 32 - DNA, 58 bp, Artificial
  M18L primer
cagtttatagttgccaatacgataacgattatacgcagcgaagcccgaatcagttccc

SEQ ID NO: 33 - DNA, 62 bp, Artificial
  M19L primer
gagagcgatgttgtcattagagcctgcgtggtcggtgttcagtttatagttgccaatacgat

SEQ ID NO: 34 - DNA, 34 bp, Artificial
  M20L primer
tcactgaacgaggagagcgatgttgtcattagag

SEQ ID NO: 35 - DNA, 26 bp, Artificial
  SpikeCTup-EcoRV primer
gatatctccggaatcaatgcgagcgt

SEQ ID NO: 36 - DNA, 35 bp, Artificial
  DI-1U/BspE I primer
tccggaatcaatgcgagcgtagtgaacatccagaa

SEQ ID NO: 37 - DNA, 48 bp, Artificial
  DI-1L primer
gcaacttcattcaaacggtcaatctctttctggatgttcactacgctc

SEQ ID NO: 38 - DNA, 45 bp, Artificial
  DI-2L primer
atcagagattcattcagatttttagcaacttcattcaaacggtca

SEQ ID NO: 39 - DNA, 51 bp, Artificial
  DI-3L primer
tcatatttgccgagttcttggaggtcaatcagagattcattcagattttta

SEQ ID NO: 40 - DNA, 43 bp, Artificial
  DI-4L primer
caaggccatttaatgtattgctcatatttgccgagttcttgga

SEQ ID NO: 41 - DNA, 50 bp, Artificial
  DI-5L primer
acctgcgatgaaacccaaccagacataccaaggccatttaatgtattgct

SEQ ID NO: 42 - DNA, 50 bp, Artificial
  DI-6L primer
cagaatagtcaccataacgatagcgatgagacctgcgatgaaacccaacc

SEQ ID NO: 43 - DNA, 50 bp, Artificial
  DI-7L primer
agagcaacaagaagtcatacaacacagcagaatagtcaccataacgatag

SEQ ID NO: 44 - DNA, 51 bp, Artificial
  DI-8L primer
daccacaagaacacgcacctttcagacaagagcaacaagaagtcatacaac

SEQ ID NO: 45 - DNA, 50 bp, Artificial
  DI-9L primer
gaatcatcctcatcaaacttacaacaagaaccacaagaacacgcaccttt

SEQ ID NO: 46 - DNA, 49 bp, Artificial
  DI-10L primer
gcttcacacccttcagaactggctcagaatcatcctcatcaaacttaca

SEQ ID NO: 47 - DNA, 35 bp, Artificial
  DI-11L primer
cctaggtataatgcagcttcacacccttcagaact

SEQ ID NO: 48 - DNA, 27 bp, Artificial
  SpikeCTdn-BstB I primer
ttcgaactaggtataatgcagcttcac

SEQ ID NO: 49 - DNA, 4800 bp, Artificial
  CoVLP cloning vector
gacggatcgggagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgctaccggactcagatcatggcagataacggcactattactgtggaggaactgaaacaactgctggaacaatggaacctcgtaatcggctttctctttctggcttggattatgttgttacagtttgcgtattctaatcgtaaccgtttcctctacattattaagctcgttttcctgtggttgttgtggcctgtaactcttgcttgctttgtgcttgctgctgtctatcgtatcaactgggttactggtggtattgctatcgctatggcttgtattgtaggcttgatgtggctgtcttatttcgttgcttctttccgtctgtttgctcgtactcgctctatgtggtcctttaatcctgagactaatatcctgctgaatgttccgctccgtggtactatcgttactagaccgctgatggaatctgaactggttattggtgccgtcattatccgtggtcatttgcgtatggctggtcactctctgggtcgttgcgatattaaggatctgccaaaggaaatcactgtagccacttctcgtactctgtcttactataaactcggtgcatcgcaacgtgtgggaactgattcgggcttcgctgcgtataatcgttatcgtattggcaactataaactgaacaccgaccacgcaggctctaatgacaacatcgctctcctcgttcagtgaaatcactagtgcggccgcaggtaccgagctcggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggcatactcttttgtgtctgaggaaactggcactctgatcgtgaactctgtactgctgtttctcgcttttgtggtattcctgctggtcactctcgctatcctcactgctcttcgtctgtgtgcctactgttgtaatatcgtgaacgtgtctctggttaagcctactgtgtatgtgtattctcgtgtgaaaaatctcaattcttctgaaggagttcccgatctgctggtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgactagtgattgatatctccggaatcaatgcgagcgtagtgaacatccagaaagagattgaccgtttgaatgaagttgctaaaaatctgaatgaacctctgattgacctccaagaactcggcaaatatgagcaatacattaaatggccttggtatgtctggttgggtttcatcgcaggtctcatcgctatcgttatggtgactattctgctgtgttgtatgacttcttgttgctcttgtctgaaaggtgcgtgttcttgtggttcttgttgtaagtttgatgaggatgattctgagccagttctgaagggtgtgaagctgcattatacctagttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaa

(19) United States
(12) Patent Application Publication
(10) Pub. No.: US 2005/0282279 A1
(43) Pub. Date: Dec. 22, 2005

(54) Expression vector encoding coronavirus-like particle

(76) Inventor: Paul Luen Hwu, Taipei (TW)

Correspondence Address: HARNESS, DICKEY & PIERCE, P.L.C., P.O. BOX 8910, RESTON, VA, 20195, US

(21) Appl. No.: 10/855,490

(22) Filed: May 28, 2004

Related U.S. Application Data
(60) Provisional application No. 60/473,924, filed on May 29, 2003.

(51) Int. Cl. C12N 15/86; C12N 7/00

(52) U.S. Cl. 435/456; 435/235.1

(57) ABSTRACT

The present invention relates to a expression vector for cloning the Class I viral fusion protein gene and as DNA vaccine candidate against virus infection diseases.

### CROSS-REFERENCE TO RELATED APPLICATIONS

This U.S. nonprovisional patent application claims priority under 35 U.S.C. § 119 of U.S. Application Ser. No. 60/473,924 filed on May 29, 2003 the entire contents of which are hereby incorporated by reference.

### FIELD OF THE INVENTION

The invention relates to the field of recombinant DNA technology, more in particular to the field of DNA vaccine. In particular the invention relates to a new expression vector encoding a virus-like particle for cloning the Class I viral fusion protein gene and as DNA vaccine candidate.

### BACKGROUND OF THE INVENTION

Vaccination has played a key role in the control of viral diseases during the past 30 years. Vaccination is based on a simple principle of immunity: once exposed to an infectious agent, an animal mounts an immune defense that protects against infection by the same agent. The goal of vaccination is to induce the animal to mount the defense prior to infection. Conventionally, this has been accomplished through the use of live attenuated or killed forms of the virus as immunogens. The success of these approaches in the past has been due in part to the presentation of native antigen and the ability of attenuated virus to elicit the complete range of immune responses obtained in natural infection. However, conventional vaccine methodologies have always been subject to a number of potential limitations. Attenuated strains can mutate to become more virulent or non-immunogenic; improperly inactivated vaccines may cause the disease that they are designed to prevent.

Recombinant DNA technology offers the potential for eliminating some of the limitations of conventional vaccines, by making possible the development of vaccines based on the use of defined antigens, rather than the intact infectious agent, as immunogens. These include peptide vaccines, consisting of chemically synthesized, immunoreactive epitopes; subunit vaccines, produced by expression of viral proteins in recombinant heterologous cells; and the use of live viral vectors for the presentation of one or a number of defined antigens. Both peptide and subunit vaccines are subject to a number of potential limitations. A major problem is the difficulty of ensuring that the conformation of the engineered proteins mimics that of the antigens in their natural environment. Suitable adjuvants and, in the case of peptides, carrier proteins, must be used to boost the immune response. In addition, these vaccines elicit primarily humoral responses, and thus may fail to evoke effective immunity.

Many different methods have been developed to introduce new genetic information into target cells. Currently, the most efficient means of introducing DNA into target cells is by employing modified viruses, so-called recombinant viral vectors. The most frequently used viral vector systems are based on retroviruses, adenoviruses, herpes viruses or the adeno-associated viruses (AAV). All systems have their specific advantages and disadvantages. Some of the vector systems possess the capacity to integrate their DNA into the host cell genome, whereas others do not. From some vector systems the viral genes can be completely removed from the vector while in other systems this is not yet possible. Some vector systems have very good in vivo delivery properties, while others do not. Some vector types are very easy to produce in large amounts, while others are very difficult to produce.

Coronaviruses carry three or four proteins in their envelopes. The M protein is the most abundant component. The small E protein is a minor but essential viral component. The importance of the S protein in pathogenesis is consistent with its biologic function in both viral entry and viral spread (Collins, A. R., et al., 1982, Virology 119:358-371; Williams, R. K., et al., 1991, Proc. Natl. Acad. Sci. USA 88:5533-5536). When expressed on the virion envelope, S protein binds to the cellular receptor and induces the fusion of viral and cell membranes during viral entry. Subsequent to infection, S protein expressed on the plasma membrane of infected cells induces cell-cell fusion. S protein also plays a role in the immune response to viral infection, as a target for neutralizing antibodies (Collins, A. R., et al., 1982, Virology 119:358-371) and as an inducer of a cell-mediated immunity (Bergmann, C. C., et al., 1996, J. Gen. Virol. 77:315-325; Castro, R. F., and S. Perlman, 1995, J. Virol. 69:8127-8131). The M and E proteins are the minimum protein units for virus assembly (Baudoux, P., et al., 1998, J. Virol. 72:8636-8643; Bos, E. C., 1996, Virology 218:52-60; de Haan, C. A. M., et al., 1998, J. Virol. 72:6838-6850; Godeke, G.-J., et al., 2000, J. Virol. 74:1566-1571; Vennema, H., et al., 1996, EMBO J. 15:2020-2028). Both are integral membrane proteins. The expression of M and E proteins together is sufficient to trigger the formation of virus-like particles (VLP). When S protein is coexpressed with M and E proteins, the S protein is incorporated into VLP with presumably authentic conformation. This has now been demonstrated for mouse hepatitis virus (MHV) (Bos, E. C., 1996, Virology 218:52-60; de Haan, C. A. M., et al., 1998, J. Virol. 72:6838-6850; Vennema, H., et al., 1996, EMBO J. 15:2020-2028), transmissible gastroenteritis virus (Baudoux, P., et al., 1998, J. Virol. 72:8636-8643), and feline infectious peritonitis virus (Godeke, G.-J., et al., 2000, J. Virol. 74:1566-1571). There is a hypothesis that the coronavirus membrane basically consists of a dense matrix of laterally interacting M proteins, which in some way requires the E protein for budding and in which the S and HE glycoproteins are incorporated, if available, by specific interactions with M via carboxyl terminal and the transmembrane region of Spike (de Haan, C. A. M., 1999, J. Virol. 73:7441-7452; Nguyen, V. -P., and B. G. Hogue. 1997, J. Virol. 71:9278-9284; Opstelten, D. -J. E., et al., 1995, J. Cell Biol. 131:339-349; Vennema, H., et al., 1996, EMBO J. 15:2020-2028). Such Spike-containing VLP can infect cells with the same infectivity like authentic virus (Bos, E. C., 1996, Virology 218:52-60). However, no effective DNA vectors are developed for use as DNA vaccine.

Therefore, there is a need to develop an effective DNA vector encoding virus-like particles that can present functional viral fusion protein in the surface of VLPs and these VLPs will be an excellent candidate as a potential vaccine against virus infection diseases.

### SUMMARY OF THE INVENTION

The present invention relates to an expression vector for cloning the Class I viral fusion protein gene and as DNA vaccine candidate, the expression vector comprising:
i) a first transcription unit comprising a membrane protein gene (M protein gene) of Coronavirus, an envelope protein gene (E protein gene) of Coronavirus and an internal ribosome entry sites (IRES) sequence, wherein the IRES is inserted into the junction of the membrane protein gene and the envelop protein gene;
ii) a first eukaryotic promoter operably linked to the membrane protein gene wherein the first promoter is located upstream of the M protein gene and drives the expression of the first transcription unit;
iii) a second transcription unit comprising a SpikeCT gene of Coronavirus and a multiple cloning site (MCS) for cloning or in-framed insertion of Class I viral fusion protein gene, wherein the MCS is located at the beginning of SpikeCT gene and has restriction enzymes cutting sites; and
iv) a second eukaryotic promoter operably linked to the SpikeCT gene wherein the second promoter is located upstream of the SpikeCT gene and drives the expression of the second transcription unit;
wherein the transcription activity of the first eukaryotic promoter is stronger than the second eukaryotic promoter.

### BRIEF DESCRIPTION OF THE DRAWING

FIG. 1 shows the plasmid map of one preferred embodiment of the expression vector of the invention.

### DETAILED DESCRIPTION OF THE INVENTION

Genes encoding viral polypeptides capable of self assembly into defective, non-self propagating viral particles can be obtained from the genomic DNA of a DNA virus or the genomic cDNA of an RNA virus or from available subgenomic clones containing the genes. These genes will include those encoding viral capsid proteins (i.e., proteins that comprise the viral protein shell) and, in the case of enveloped viruses, such as retroviruses, the genes encoding viral envelope glycoproteins. The virus-like particles may be isolated and used themselves as immunogens for vaccination against pathogenic viruses, or for therapeutic purposes, such as enhancing immune responses in an infected individual, or for targeted delivery of therapeutic agents, such as cytotoxic drugs, to specific cell types.

The present invention provides an expression vector for cloning the Class I viral fusion protein gene and as DNA vaccine candidate, the expression vector comprising:
i) a first transcription unit comprising a membrane protein gene (M protein gene) of Coronavirus, an envelope protein gene (E protein gene) of Coronavirus and an internal ribosome entry sites (IRES) sequence, wherein the IRES is inserted into the junction of the membrane protein gene and the envelop protein gene;
ii) a first eukaryotic promoter operably linked to the membrane protein gene wherein the first promoter is located upstream of the M protein gene and drives the expression of the first transcription unit;
iii) a second transcription unit comprising a SpikeCT gene of Coronavirus and a multiple cloning site (MCS) for cloning or in-framed insertion of Class I viral fusion protein gene, wherein the MCS is located at the beginning of SpikeCT gene and has restriction enzymes cutting sites; and
iv) a second eukaryotic promoter operably linked to the SpikeCT gene wherein the second promoter is located upstream of the SpikeCT gene and drives the expression of the second transcription unit;
wherein the transcription activity of the first eukaryotic promoter is stronger than the second eukaryotic promoter.

According to the invention, the expression vector comprises a first transcription unit comprising a membrane protein gene (M protein gene) of Coronavirus, an envelope protein gene (E protein gene) of Coronavirus and an internal ribosome entry sites (IRES) sequence, wherein the IRES is inserted into the junction of the membrane protein gene and the envelop protein gene.

According to the invention, the envelope protein gene of the invention encodes envelope protein (E protein) of Coronavirus. The E protein is a small, envelope-associated protein. The E protein is a minor but essential viral component. In cells, it accumulates in and induces the coalescence of the membranes of the intermediate compartment (IC), giving rise to typical structures. A fraction of the proteins appear extracellularly in membranous structures of unknown identity. Preferably, the coronvirus of the invention is pig, human or avian coronavirus. More preferably, the coronavirus is pigTGEV coronavirus, human 229E coronavirus or human SARS virus. More preferably, the E protein gene of the invention has the sequence as set forth in SEQ ID NO:1.

The coding sequence of SARS E gene (SEQ ID NO:1)

ATGGCATACT CTTTTGTGTC TGAGGAAACT GGCACTCTGA
TCGTGAACTC
TGTACTGCTG
TTTCTCGCTT TTGTGGTATT CCTGCTGGTC ACTCTCGCTA
TCCTCACTGC
TCTTCGTCTG
TGTGCCTACT GTTGTAATAT CGTGAACGTG TCTCTGGTTA
AGCCTACTGT
GTATGTGTAT
TCTCGTGTGA AAAATCTCAA TTCTTCTGAA GGAGTTCCCG
ATCTGCTGGT
CTAG

According to the invention, the membrane protein gene of the invention encodes membrane protein (M protein) of Coronavirus. The M protein is the most abundant component; it is a type III glycoprotein consisting of a short amino-terminal ectodomain, three successive transmembrane domains, and a long carboxy-terminal domain on the inside of the virion (or in the cytoplasm). Preferably, the coronvirus of the invention is pig, human or avian coronvirus. More preferably, the coronavirus is pig TGEV coronavirus, human 229E coronavirus or human SARS virus. More preferably, the M protein gene of the invention has the sequence as set forth in SEQ ID NO:2.

The coding sequence of SARS M gene (SEQ ID NO:2)

ATGGCAGATA ACGGCACTAT TACTGTGGAG GAACTGAAAC
AACTGCTGGA
ACAATGGAAC
CTCGTAATCG GCTTTCTCTT TCTGGCTTGG ATTATGTTGT
TACAGTTTGC
GTATTCTAAT
CGTAACCGTT TCCTCTACAT TATTAAGCTC GTTTTCCTGT
GGTTGTTGTG
GCCTGTAACT

5′ ends of one or more additional coding sequences which are then inserted into the vectors at the end of the original coding sequence, so that the coding sequences are separated from one another by IRES. According to the invention, any IRES derivatives also can be used in the plasmid construct of the invention. The IRES sequence allows the ribosomal machinery to initiate translation from a secondary site, within a single transcript.

According to the invention, the expression vector of the invention comprises a first eukaryotic promoter operably linked to the membrane protein gene wherein the first promoter is located upstream of the M protein gene and drives the expression of the first transcription unit. Preferably, the first eukaryotic promoter is viral derived promoter like CMV, SV40, RSV, HIV-1 LTR and the hybrid Beta-actin/CMV promoter enhancer and muscle-specific desmin, creatine kinase, beta-Actin promoter and other ubiquitous expression promoter like EF1alpha, Ubiquitin promoter. The most preferably, the first eukaryotic promoter is pCMV, Hybrid Beta-actin/CMV promoter enhancer, beta-Actin promoter.

According to the invention, a second transcription unit comprises a SpikeCT gene and a multiple cloning site (MCS) having restriction enzymes cutting sites, wherein the MCS is located at the beginning of SpikeCT gene. The SpikeCT gene interacts with the M protein interaction domain, which encodes C-terminal heptad repeat located upstream of an aromatic residue-rich region juxtaposed to the transmembrane segment of Spike protein of coronavirus. The multiple cloning site (MCS) has several restriction enzymes cutting sites. The restriction enzyme cutting site includes, but not limited to, SmaI, BsaB I, EcoR V and BspE I. The MCS is located at the beginning of SpikeCT gene and can be used for cloning or in-framed insertion of Class I viral fusion protein gene. The Class I viral fusion protein gene includes, but not limited to, gp160 of HIV, HA of influenza virus and Spike of SARS virus. The Class I viral fusion protein gene is derived form the viruses that adapt Class I viral fusion mechanism comprises coronavirus, HIV, and influenza virus, into cloning/expression vector of the invention and then used as DNA vaccine candidate. Preferably, the SpikeCT gene having the sequence as set forth in SED ID No:3.

The coding sequence of SpikeCT gene (SEQ ID NO:3)

GATATCTCCGGA ATCAATGCGA GCGTAGTGAA CATCCAGAAA
GAGATTGACC GTTTGAATGA AGTTGCTAAA AATCTGAATG
AACCTCTGAT
TGACCTCCAA
GAACTCGGCA AATATGAGCA ATACATTAAA TGGCCTTGGT
ATGTCTGGTT
GGGTTTCATC
GCAGGTCTCA TCGCTATCGT TATGGTGACT ATTCTGCTGT
GTTGTATGAC
TTCTTGTTGC
TCTTGTCTGA AAGGTGCGTG TTCTTGTGGT TCTTGTTGTA
AGTTTGATGA
GGATGATTCT
GAGCCAGTTC TGAAGGGTGT GAAGCTGCAT TATACCTAGT TCGAA
CTTGCTTGCT TTGTGCTTGC TGCTGTCTAT CGTATCAACT
GGGTTACTGG
TGGTATTGCT
ATCGCTATGG CTTGTATTGT AGGCTTGATG TGGCTGTCTT
ATTTCGTTGC
TTCTTTCCGT
CTGTTTGCTC GTACTCGCTC TATGTGGTCC TTTAATCCTG
AGACTAATAT
CCTGCTGAAT
GTTCCGCTCC GTGGTACTAT CGTTACTAGA CCGCTGATGG
AATCTGAACT
GGTTATTGGT
GCCGTCATTA TCCGTGGTCA TTTGCGTATG GCTGGTCACT
CTCTGGGTCG
TTGCGATATT
AAGGATCTGC CAAAGGAAAT CACTGTAGCC ACTTCTCGTA
CTCTGTCTTA
CTATAAACTC
GGTGCATCGC AACGTGTGGG AACTGATTCG GGCTTCGCTG
CGTATAATCG
TTATCGTATT
GGCAACTATA AACTGAACAC CGACCACGCA GGCTCTAATG
ACAACATCGC
TCTCCTCGTT
CAGTGA

According to the invention, for assembly of the coronavirus envelope, only the M protein and the E protein are needed (Cornelis A. M. de Haan et al., Journal of Virology, June 2000, p. 4967-4978). Expression in cells of the genes coding for these proteins leads to the formation and release of viruslike particles (VLPs) similar in size and shape to authentic virions.

According to the invention, the first transcription unit comprises an internal ribosome entry sites (IRES) sequence that is inserted into the junction of M gene and E gene. The IRES allows the translation of two or more proteins from a di- or polycistronic mRNA. The IRES units are fused to

According to the invention, the expression vector of the invention can produce a virus-like particle and express the functional fusion gene of the Class I viruses on the surface of virus-like particle. Thus, there are several restriction enzymes cutting sites (multiple cloning site, MCS) including, but not limiting to, SmaI, BsaB I, EcoR V, BspE I located at the beginning of “SpikeCT” gene and these sites can be used for insertion or cloning Class I viral fusion protein gene into the cloning/expression vector of the invention and then used as DNA vaccine candidate. The viruses that adapt Class I viral fusion mechanism includes, but not limited to, coronavirus, HIV, influenza virus, and so on.

According to the invention, the second promoter is located upstream of the SpikeCT gene and drives the expression of the second transcription unit. Preferably, the second eukaryotic promoter is viral derived promoter like CMV, SV40, RSV, HIV-1 LTR and the hybrid Beta-actin/CMV promoter enhancer and muscle-specific desmin, creatine kinase, beta-Actin promoter and other ubiquitous orcontitutive expression promoter like EF1alpha, Ubiquitin promoter. The most preferably, the second eukaryotic promoter is pCMV, SV40, beta-Actin and EF1alpha promoter.

According to the invention, the transcription units of the invention comprise a polyA sigal. Persons skilled in the art can recognize that any transcription has the polyA signal. Preferably, the transcription units of the invention comprises BGH polyA signal.

According to the invention, the expression vector further comprises a replication origin for the propagation plasmid in a host cell. The determination of the replication origin depends on the types of the host cells.

According to the invention, the expression vector of the invention further comprises an antibiotics-resistant gene as the selection marker.

According to the invention, the expression vector of the invention can spontaneously form virus-like particles. The virus-like particles can be used as vaccine candidate against virus infection. The virus-like particle lacks the genetic materials of the virus. Therefore, such particles do not have infectious ability. The expression vector of the invention can be administrated to animals and transfected with host cells to produce the virus-like particles.

Most viruses consist of few proteins only with structural restrictions impinged on them. In fact, using these few proteins, viruses are forced to generate a quasi-crystalline, highly repetitive surface. Such highly repetitive antigens efficiently cross-link B cell receptors, a process which generates strong activation signal in B cells. In contrast, self-antigens are usually not highly organized, in particular not those accessible to B cells. Thus, B cells use antigen organization as a marker for infectious non-self. Vaccines based on virus like particles (VLPs) exploit this basic phenomenon, since VLPs exhibit surfaces as repetitive and organized as those of viruses. Consequently, similar to viruses, VLPs are able to trigger strong B cell responses in the absence of adjuvants. Based on the above principle, the virus-like particles produced through the expression of the expression vector of the invention in the cells can be used as a vaccine candidate against virus diseases.

### EXAMPLE

E Gene Synthesized by PCR

The polymerase chain reaction (PCR) was used to synthesize the E gene of SARS virus. The PCR template(0.1 pmole) of SARS E gene is the mixture of primers that listed below and PCR reaction was performed using the standard PCR method described by Innis et al. (PCR protocols. A guide to methods and applications, 1990, Academic Press) using KOD Taq polymerase (Novagene.com). The PCR primers are shown below:

| | |
|---|---|
| 5′-ACC ATG GCA TAC TCT TTT GTG TCT | (SEQ ID NO: 4) |
| GAG GAA ACT G-3′* | |
| E1L | |
| 5′-ACA GCA GTA CAG AGT TCA CGA TCA | (SEQ ID NO: 5) |
| GAG TGC CAG TTT CCT CAG ACA CAA | |
| AAG-3′ | |
| E2L | |
| 5′-TGA CCA GCA GGA ATA CCA CAA AAG | (SEQ ID NO: 6) |
| CGA GAA ACA GCA GTA CAG AGT TCA | |
| CGA-3′ | |
| E3L | |
| 5′-GCA CAC AGA CGA AGA GCA GTG AGG | (SEQ ID NO: 7) |
| ATA GCG AGA GTG ACC AGC AGG AAT | |
| ACC ACA-3′ | |
| E4L | |
| 5′-ACC AGA GAC ACG TTC ACG ATA TTA | (SEQ ID NO: 8) |
| CAA CAG TAG GCA CAC AGA CGA AGA | |
| GCA G-3′ | |
| E5L | |
| 5′-GAT TTT TCA CAC GAG AAT ACA CAT | (SEQ ID NO: 9) |
| ACA CAG TAG GCT TAA CCA GAG ACA | |
| CGT TCA CGA T-3′* | |
| E6L | |
| 5′-TCT AGA CCA GCA GAT CGG GAA CTC | (SEQ ID NO: 10) |
| CTT CAG AAG AAT TGA GAT TTT TCA | |
| CAC GAG AAT ACA CA-3′ | |
| E7L | |
| 5′-TCT AGA CCA GCA GAT CGG GA-3′ | (SEQ ID NO: 11) |

The DNA template is initial denaturized at: 95° C. for 3 minutes. The PCR conditions are listed as follow:

First PCR reaction with 10 cycles of 3 steps
1. Annealing: 58° C. for 20 seconds
2. Extension: 72° C. for 40 seconds
3. Denaturation: 95° C. for 1 minutes

Second PCR reaction with 20 cycles of 3 steps
1. Denaturation: 95° C. for 1 minutes
2. Annealing: 62° C. for 20 seconds
3. Extension: 72° C. for 40 seconds

The resultant double-stranded full-length of E gene products were analyzed on 1.2% agarose gel and purified with QIAquick PCR purification kit (Qiagen Inc.) and then ligated with pGEM-T vector (Promega Co.) to obtain pGEM(T)/E^{A+} clones. The sequence of E gene is as set forth in SEQ ID NO: 1.

M Gene Synthesized by Splicing-Over PCR

The synthesis of M gene of SARS virus was similar to the PCR method as mentioned in the above and the PCR template(0.1 pmole) of SARS M gene is the mixture of primers that listed below by using KOD Taq polymerase (Novagene.com) are as follows:

The PCR primers are listed as follows:

| | |
|---|---|
| M1-1U | |
| 5′-TGA TCA TGG CAG ATA ACG GCA | (SEQ ID NO: 12) |
| C-3′ | |
| M1U | |
| 5′-TGA TCA TGG CAG ATA ACG GCA CTA | (SEQ ID NO: 13) |
| TTA CTG TGG AGG A-3′ | |
| M1L | |
| 5′-GTT CCA TTG TTC CAG CAG TTG TTT | (SEQ ID NO: 14) |
| CAG TTC CTC CAC AGT AAT AGT GCC | |
| G-3′ | |
| M2L | |
| 5′-AAG CCA GAA AGA GAA AGC CGA TTA | (SEQ ID NO: 15) |
| CGA GGT TCC ATT GTT CCA GCA GTT | |
| G-3′ | |
| M3L | |
| 5′-AGA ATA CGC AAA CTG TAA CAA CAT | (SEQ ID NO: 16) |
| AAT CCA AGC CAG AAA GAG AAA GCC | |
| GA-3′ | |
| M4L | |
| 5′-CGA GCT TAA TAA TGT AGA GGA AAC | (SEQ ID NO: 17) |
| GGT TAC GAT TAG AAT ACG CAA ACT | |
| GTA ACA ACA-3′ | |
| M5L | |
| 5′-CAA GAG TTA CAG GCC ACA ACA ACC | (SEQ ID NO: 18) |
| ACA GGA AAA CGA GCT TAA TAA TGT | |
| AGA GGA AA-3′ | |
| M6L | |
| 5′-TTG ATA CGA TAG ACA GCA GCA AGC | (SEQ ID NO: 19) |
| ACA AAG CAA GCA AGA GTT ACA GGC | |
| CAC AAC A-3′ | |
| M7L | |
| 5′-CAA GCC ATA GCG ATA GCA ATA CCA | (SEQ ID NO: 20) |
| CCA GTA ACC CAG TTG ATA CGA TAG | |
| ACA GCA GCA A-3′ | |
| M8L | |
| 5′-AAC GAA ATA AGA CAG CCA CAT CAA | (SEQ ID NO: 21) |
| GCC TAC AAT ACA AGC CAT AGC GAT | |
| AGC AAT AC-3′ | |
| M9L | |
| 5′-ACA TAG AGC GAG TAC GAG CAA ACA | (SEQ ID NO: 22) |
| GAC GGA AAG AAG CAA CGA AAT AAG | |
| ACA GCC ACA TC-3′ | |
| M10U | |
| 5′-TCC TGC TGA ATG TTC CGC TC-3′ | (SEQ ID NO: 23) |
| M10L | |
| 5′-GAG CGG AAC ATT CAG CAG GAT ATT | (SEQ ID NO: 24) |
| AGT CTC AGG ATT AAA GGA CCA CAT | |
| AGA GCG AGT ACG AGC AA-3′ | |
| M11L | |
| 5′-CAT CAG CGG TCT AGT AAC GAT AGT | (SEQ ID NO: 25) |
| ACC ACG GAG CGG AAC ATT CAG CAG | |
| GA-3′ | |
| M12L | |
| 5′-ACC ACG GAT AAT GAC GGC ACC AAT | (SEQ ID NO: 26) |
| AAC CAG TTC AGA TTC CAT CAG CGG | |
| TCT AGT AAC GAT-3′ | |
| M13L | |
| 5′-ATA CGC AAA TGA CCA CGG ATA ATG | (SEQ ID NO: 27) |
| ACG GCA C-3′ | |
| M14L | |
| 5′-AAC GAC CCA GAG AGT GAC CAG CCA | (SEQ ID NO: 28) |
| TAC GCA AAT GAC CAC GGA TAA-3′ | |
| M15L | |
| 5′-TAC AGT GAT TTC CTT TGG CAG ATC | (SEQ ID NO: 29) |
| CTT AAT ATC GCA ACG ACC CAG AGA | |
| GTG-3′ | |
| M16L | |
| 5′-CCG AGT TTA TAG TAA GAC AGA GTA | (SEQ ID NO: 30) |
| CGA GAA GTG GCT ACA GTG ATT TCC | |
| TTT GGC AGA-3′ | |
| M17L | |
| 5′-CGA AGC CCG AAT CAG TTC CCA CAC | (SEQ ID NO: 31) |
| GTT GCG ATG CAC CGA GTT TAT AGT | |
| AAG ACA GAG T-3′ | |
| M18L | |
| 5′-CAG TTT ATA GTT GCC AAT ACG ATA | (SEQ ID NO: 32) |
| ACG ATT ATA CGC AGC GAA GCC CGA | |
| ATC AGT TCC C-3′ | |
| M19L | |
| 5′-GAG AGC GAT GTT GTC ATT AGA GCC | (SEQ ID NO: 33) |
| TGC GTG GTC GGT GTT CAG TTT ATA | |
| GTT GCC AAT ACG AT-3′ | |
| M20L | |
| 5′-TCA CTG AAC GAG GAG AGC GAT GTT | (SEQ ID NO: 34) |
| GTC ATT AGA G-3′ | |

The PCR conditions are essentially as mentioned above. The resultant double-stranded full-length of M gene products were analyzed on 1.0% agarose gel and purified with QIAquick PCR purification or gel extraction kit (Qiagen Inc.) and then ligated with pGEM-T vector (Promega Co.) to obtain pGEM(T)/M clones. The sequence of M gene is as set forth in SEQ ID NO: 2.

The “SpikeCT” gene Encoding a heptad region 2 and transmembrane domain of Spike Protein Gene of SARS Virus Synthesized by PCR.

The PCR method and condition, purification and cloning of the PCR products are as mentioned above. The DNA template (0.1 pmole) of SpikeCT gene is the mixture of primers listed below:

The PCR primers are listed as follows:

| | |
|---|---|
| SpikeCTup-EcoRV | |
| 5′-GAT ATC TCC GGA ATC AAT GCG AGC | (SEQ ID NO: 35) |
| GT-3′ | |
| DI-1U/BspE I | |
| 5′-5′-TCC GGA ATC AAT GCG AGC GTA | (SEQ ID NO: 36) |
| GTG AAC ATC CAG AA-3′ | |
| DI-1L | |
| 5′-GCA ACT TCA TTC AAA CGG TCA ATC | (SEQ ID NO: 37) |
| TCT TTC TGG ATG TTC ACT ACG CTC-3′ | |
| DI-2L | |
| 5′-ATC AGA GAT TCA TTC AGA TTT TTA | (SEQ ID NO: 38) |
| GCA ACT TCA TTC AAA CGG TCA-3′ | |
| DI-3L | |
| 5′-TCA TAT TTG CCG AGT TCT TGG AGG | (SEQ ID NO: 39) |
| TCA ATC AGA GAT TCA TTC AGA TTT | |
| TTA-3′ | |
| DI-4L | |
| 5′-CAA GGC CAT TTA ATG TAT TGC TCA | (SEQ ID NO: 40) |
| TAT TTG CCG AGT TCT TGG A-3′ | |
| DI-5L | |
| 5′-ACC TGC GAT GAA ACC CAA CCA GAC | (SEQ ID NO: 41) |
| ATA CCA AGG CCA TTT AAT GTA TTG | |
| CT-3′ | |
| DI-6L | |
| 5′-CAG AAT AGT CAC CAT AAC GAT AGC | (SEQ ID NO: 42) |
| GAT GAG ACC TGC GAT GAA ACC CAA | |
| CC-3′ | |
| DI-7L | |
| 5′-AGA GCA ACA AGA AGT CAT ACA ACA | (SEQ ID NO: 43) |
| CAG CAG AAT AGT CAC CAT AAC GAT | |
| AG-3′ | |
| DI-8L | |
| 5′-ACC ACA AGA ACA CGC ACC TTT CAG | (SEQ ID NO: 44) |
| ACA AGA GCA ACA AGA AGT CAT ACA | |
| AC-3′ | |
| DI-9L | |
| 5′-GAA TCA TCC TCA TCA AAC TTA CAA | (SEQ ID NO: 45) |
| CAA GAA CCA CAA GAA CAC GCA CCT | |
| TT-3′ | |
| DI-10L | |
| 5′-GCT TCA CAC CCT TCA GAA CTG GCT | (SEQ ID NO: 46) |
| CAG AAT CAT CCT CAT CAA ACT TAC | |
| A-3′ | |
| DI-11L | |
| 5′-CCT AGG TAT AAT GCA GCT TCA CAC | (SEQ ID NO: 47) |
| CCT TCA GAA CT-3′ | |
| SpikeCTdn-BstB I | |
| 5′-TTC GAACT AGG TAT AAT GCA GCT | (SEQ ID NO: 48) |
| TCA C-3′ | |

The PCR conditions are as mentioned above. The resultant double-stranded SCT320 gene products were purified with Qiagen/PCR purification kit (Qiagen Inc.) and cloned with pGEM-T vector (Promega Co.) to obtain pGEM(T)/SpikeCT(EcoRV/BstBI). The pGEM(T)/SpikeCT (EcoRV/BstBI) plasmid in which containing a gene encodes C-terminal heptad repeat located upstream of an aromatic residue-rich region juxtaposed to the transmembrane segment of Spike protein of SARS coronavirus. The coding sequence of “SCT320” gene is as set forth in SEQ ID NO: 3.

Construction of Expression vector of the Invention

The manipulation of DNA is instructed by Sambrook and Russell et al ┌Molecular cloning third edition┘, Cold Spring Harbor Laboratory Express. The restriction enzymes, T4DNA ligase, Klenow enzyme are purchased from New England Biolab.com and performed by manufacture's recommendation.

I. Construction of pCMV Promoter-Driven SARS N Gene Plasmid

The pGEM(T)/M clone was used as DNA template for PCR with T7 and SP6 promoter primer, and the PCR condition, except the Annealing temperature was set at 50° C. for 20 seconds, are same as mentioned above. The resulting M gene PCR products were purified with QIAquick PCR purification kit (Qiagen Inc.) and digested with Bcl I and NotI to obtain DNA inserts containing SARS M gene. The pEGFP-N1 plasmid (Clontech Co.) was digested with BglII and Not I and the resulting Bgl II, Not I digested pEGFP-N1 plasmid were uesd as DNA vector for ligation with Bcl I, Not I digested SARS M gene DNA inserts to obtain pN1/M DNA plasmid. The pN1/M DNA plasmid was digested with NheI and Not I to obtain DNA inserts containing SARS M gene. The pACT plasmid (Promega Co.) was digested with Nhe I and Not I. The resulting Nhe I, Not I digested pACT DNA vector were ligated with Nhe I, Not I digested SARS M gene DNA inserts to obtain pACT/M DNA plasmid. The pACT/M DNA plasmid was digested with Bgl II and Asp 718 to obtain DNA inserts containing CMV promoter and SARS M gene. The pCDNA3.1(+) plasmid (Invitrogen Co.) was digested with BglII and Asp718. The resulting BglII, Asp718 digested pCDNA3.1(+)DNA vector were ligated with BglII, Asp718 digested DNA containing CMV promoter and SARS M gene inserts to obtain pCDNA 3.1+M DNA plasmid.

II. Constructing IRES-SARS E Gene Plasmid

The pIRES2-EGFP (Clontech Co.) was digested with Xho I and Nco I to get “IRES” DNA insert. The GL3basic vector (Promega Co.) was digested with Xho I and Nco I. The Xho I,Nco I digested pGL3basic DNA vector were ligated with Xho I,Nco I digested IRES DNA insert by using T4 DNA ligase (NEB Co.). The ligation mix was transformed into E. coli DH5 α competent cells to obtain pGL3B/IRES-luciferase DNA plasmid. The T/E^{A+} clone, containing SARS E gene with second Alanine insertion mutant, was digested with NcoI and XbaI to obtain SARS E gene DNA inserts. The pGL3B/IRES-luciferase DNA plasmid was digested with NcoI and XbaI. The Nco I, Xba I digested pGL3B/IRES-luciferase DNA vector was ligated with Nco I, Xba I digested SARS E^{A+} gene DNA inserts to get pGL3B/IRES-E^{A+} DNA plasmid.

III. Construction of pCMV-Driven M+E Gene Expression Plasmid (CoronaVirus-Like Particle (CoVLP) Expression Vector)

The pGL3B/IRES-E^{A+} DNA plasmid was digested with BamHI and XbaI to obtain DNA inserts containing IRES and SARS E^{A+} gene. The pCDNA3.1+M DNA plasmid was digested with BamHI and XbaI. The resultant BamH I, Xba I digested pCDNA3.1+M DNA vector were ligated with BamH I, Xba I digested DNA inserts containing IRES and SARS E^{A+} fused gene to obtain pCDNA3.1+M/IRES-E^{A+} plasmid.

IV. Constructing the DNA vector of invention

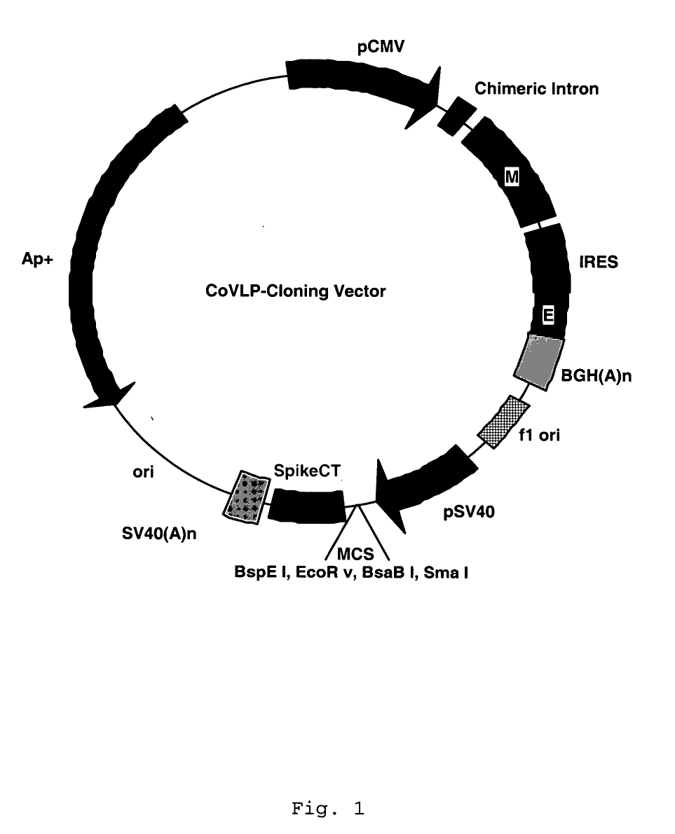

The pGEM(T)/SpikeCT (EcoRV/BstBI) plasmid, in which containing a gene encodes C-terminal heptad repeat located upstream of an aromatic residue-rich region juxtaposed to the transmembrane segment of Spike protein of SARS coronavirus, was digested with Spe I and filled-in by Klenow enzyme and dNTPs. After separated onto 1.2% agarose gel, the DNA fragment were purified with QIAquick gel extraction kit (QIAGEN. Com.) and further digested with BstB I. The resultant “SpikeCT” gene fragments were used as DNA inserts and ligated with BsaB I, BstB I digested, gel purified pCDNA3.1+M/IRES-E^{A+} DNA vector plasmid. After transformation into E. coli DH5alpha cells, get the DNA vector of invention, so-called CoVLP-cloning vector. There are several restriction enzymes cutting sites (multiple cloning site, MCS) including SmaI, BsaB I, EcoR V, BspE I located at the beginning of “SpikeCT” gene and these sites can be used for cloning or in-frame fused Class I viral fusion protein gene with SpikeCT gene in the vector of invention and then the resultant DNA plasmid can be used as DNA vaccine candidate. The map of the CoVLP-cloning vector is shown in FIG. 1. The sequence of the CoVLP-cloning vector is as set forth in SEQ ID NO:49.

The DNA sequence of CoVLP cloning vector (pCDNA3.1+M+IRES−E+SpikeCT) (SEQ ID NO:49)

GACGGATCGGGagatctTCAATATTGGCCATTAGCCATATTATTCATTGGTTATATAG
CATAAATCAATATTGGCTATTG
GCCATTGCATACGTTGTATCTATATCATAATATGTACATTTATATTGGCTCATGTC
CAATATGACCGCCATGTTGGCATT
GATTATTGACTAGTTATTAATAGTAATCAATTACGGGGTCATTAGTTCATAGCCC
ATATATGGAGTTCCGCGTTACATAA
CTTACGGTAAATGGCCCGCCTGGCTGACCGCCCAACGACCCCCGCCCATTGACGT
CAATAATGACGTATGTTCCCATAGT
AACGCCAATAGGGACTTTCCATTGACGTCAATGGGTGGAGTATTTACGGTAAACT
GCCCACTTGGCAGTACATCAAGTGT
ATCATATGCCAAGTCCGCCCCCTATTGACGTCAATGACGGTAAATGGCCCGCCTG
GCATTATGCCCAGTACATGACCTTA
CGGGACTTTCCTACTTGGCAGTACATCTACGTATTAGTCATCGCTATTACCATGGT
GATGCGGTTTTGGCAGTACACCAA
TGGGCGTGGATAGCGGTTTGACTCACGGGGATTTCCAAGTCTCCACCCCATTGAC
GTCAATGGGAGTTTGTTTTGGCACC
AAAATCAACGGGACTTTCCAAAATGTCGTAACAACTGCGATCGCCCGCCCCGTTG
ACGCAAATGGGCGGTAGGCGTGTAC
GGTGGGAGGTCTATATAAGCAGAGCTCGTTTAGTGAACCGTCAGATCACTAGAA
GCTTTATTGCGGTAGTTTATCACAGT
TAAATTGCTAACGCAGTCAGTGCTTCTGACACAACAGTCTCGAACTTAAGCTGCA
GTGACTCTCTTAAGGTAGCCTTGCA
GAAGTTGGTCGTGAGGCACTGGGCAGGTAAGTATCAAGGTTACAAGACAGGTTT
AAGGAGACCAATAGAAACTGGGCTTG
TCGAGACAGAGAAGACTCTTGCGTTTCTGATAGGCACCTATTGGTCTTACTGACA
TCCACTTTGCCTTTCTCTCCACAGG
TGTCCACTCCCAGTTCAATTACAGCTCTTAAGGCTAGAGTACTTAATACGACTCA
CTATAGGCTAGCGCTACCGGACTCA
GATCATGGCAGATAACGGCACTATTACTGTGGAGGAACTGAAACAACTGCTGGA
ACAATGGAACCTCGTAATCGGCTTTC
TCTTTCTGGCTTGGATTATGTTGTTACAGTTTGCGTATTCTAATCGTAACCGTTTC
CTCTACATTATTAAGCTCGTTTTC
CTGTGGTTGTTGTGGCCTGTAACTCTTGCTTGCTTTGTGCTTGCTGCTGTCTATCGT
ATCAACTGGGTTACTGGTGGTAT
TGCTATCGCTATGGCTTGTATTGTAGGCTTGATGTGGCTGTCTTATTTCGTTGCTT
CTTTCCGTCTGTTTGCTCGTACTC
GCTCTATGTGGTCCTTTAATCCTGAGACTAATATCCTGCTGAATGTTCCGCTCCGT
GGTACTATCGTTACTAGACCGCTG
ATGGAATCTGAACTGGTTATTGGTGCCGTCATTATCCGTGGTCATTTGCGTATGG
CTGGTCACTCTCTGGGTCGTTGCGA
TATTAAGGATCTGCCAAAGGAAATCACTGTAGCCACTTCTCGTACTCTGTCTTAC
TATAAACTCGGTGCATCGCAACGTG
TGGGAACTGATTCGGGCTTCGCTGCGTATAATCGTTATCGTATTGGCAACTATAA
ACTGAACACCGACCACGCAGGCTCT
AATGACAACATCGCTCTCCTCGTTCAGTGAAATCACTAGTGCGGCCGCAGGTACC
GAGCTCGGATCCGCCCCTCTCCCTC
CCCCCCCCCTAACGTTACTGGCCGAAGCCGCTTGGAATAAGGCCGGTGTGCGTTT
GTCTATATGTTATTTTCCACCATAT
TGCCGTCTTTTGGCAATGTGAGGGCCCGGAAACCTGGCCCTGTCTTCTTGACGAG
CATTCCTAGGGGTCTTTCCCCTCTC
GCCAAAGGAATGCAAGGTCTGTTGAATGTCGTGAAGGAAGCAGTTCCTCTGGAA
GCTTCTTGAAGACAAACAACGTCTGT
AGCGACCCTTTGCAGGCAGCGGAACCCCCCACCTGGCGACAGGTGCCTCTGCGG
CCAAAAGCCACGTGTATAAGATACAC
CTGCAAAGGCGGCACAACCCCAGTGCCACGTTGTGAGTTGGATAGTTGTGGAAA
GAGTCAAATGGCTCTCCTCAAGCGTA
TTCAACAAGGGGCTGAAGGATGCCCAGAAGGTACCCCATTGTATGGGATCTGAT
CTGGGGCCTCGGTGCACATGCTTTAC
ATGTGTTTAGTCGAGGTTAAAAAAACGTCTAGGCCCCCCGAACCACGGGGACGT
GGTTTTCCTTTGAAAAACACGATGAT
AATATGGCCACAACCATGGCATACTCTTTTGTGTCTGAGGAAACTGGCACTCTGA
TCGTGAACTCTGTACTGCTGTTTCT
CGCTTTTGTGGTATTCCTGCTGGTCACTCTCGCTATCCTCACTGCTCTTCGTCTGTG
TGCCTACTGTTGTAATATCGTGA
ACGTGTCTCTGGTTAAGCCTACTGTGTATGTGTATTCTCGTGTGAAAAATCTCAAT
TCTTCTGAAGGAGTTCCCGATCTG
CTGGTCTAGAGGGCCCGTTTAAACCCGCTGATCAGCCTCGACTGTGCCTTCTAGT
TGCCAGCCATCTGTTGTTTGCCCCT
CCCCCGTGCCTTCCTTGACCCTGGAAGGTGCCACTCCCACTGTCCTTTCCTAATAA
AATGAGGAAATTGCATCGCATTGT
CTGAGTAGGTGTCATTCTATTCTGGGGGGTGGGGTGGGGCAGGACAGCAAGGGG
GAGGATTGGGAAGACAATAGCAGGCA
TGCTGGGGATGCGGTGGGCTCTATGGCTTCTGAGGCGGAAAGAACCAGCTGGGG
CTCTAGGGGGTATCCCCACGCGCCCT
GTAGCGGCGCATTAAGCGCGGCGGGTGTGGTGGTTACGCGCAGCGTGACCGCTA
CACTTGCCAGCGCCCTAGCGCCCGCT
CCTTTCGCTTTCTTCCCTTCCTTTCTCGCCACGTTCGCCGGCTTTCCCCGTCAAGCT
CTAAATCGGGGGCTCCCTTTAGG
GTTCCGATTTAGTGCTTTACGGCACCTCGACCCCAAAAAACTTGATTAGGGTGAT
GGTTCACGTAGTGGGCCATCGCCCT
GATAGACGGTTTTTCGCCCTTTGACGTTGGAGTCCACGTTCTTTAATAGTGGACTC
TTGTTCCAAACTGGAACAACACTC
AACCCTATCTCGGTCTATTCTTTTGATTTATAAGGGATTTTGCCGATTTCGGCCTA
TTGGTTAAAAAATGAGCTGATTTA
ACAAAAATTTAACGCGAATTAATTCTGTGGAATGTGTGTCAGTTAGGGTGTGGAA
AGTCCCCAGGCTCCCCAGCAGGCAG
AAGTATGCAAAGCATGCATCTCAATTAGTCAGCAACCAGGTGTGGAAAGTCCCC
AGGCTCCCCAGCAGGCAGAAGTATGC
AAAGCATGCATCTCAATTAGTCAGCAACCATAGTCCCGCCCCTAACTCCGCCCAT
CCCGCCCCTAACTCCGCCCAGTTCC
GCCCATTCTCCGCCCCATGGCTGACTAATTTTTTTTATTTATGCAGAGGCCGAGGC
CGCCTCTGCCTCTGAGCTATTCCA
GAAGTAGTGAGGAGGCTTTTTTGGAGGCCTAGGCTTTTGCAAAAAGCTCCCGGG
AGCTTGTATATCCATTTTCGGATCTG
ATCAAGAGACAGGATGActagtgattgatatcTCCGGAATCAATGCGAGCGTAGTGAACA
TCCAGAAAGAGATTGACCGT
TTGAATGAAGTTGCTAAAAATCTGAATGAACCTCTGATTGACCTCCAAGAACTCG
GCAAATATGAGCAATACATTAAATG
GCCTTGGTATGTCTGGTTGGGTTTCATCGCAGGTCTCATCGCTATCGTTATGGTGA
CTATTCTGCTGTGTTGTATGACTT
CTTGTTGCTCTTGTCTGAAAGGTGCGTGTTCTTGTGGTTCTTGTTGTAAGTTTGAT
GAGGATGATTCTGAGCCAGTTCTG
AAGGGTGTGAAGCTGCATTATACCTAGttcgaaATGACCGACCAAGCGACGCCCAAC
CTGCCATCACGAGATTTCGATTC
CACCGCCGCCTTCTATGAAAGGTTGGGCTTCGGAATCGTTTTCCGGGACGCCGGC
TGGATGATCCTCCAGCGCGGGGATC
TCATGCTGGAGTTCTTCGCCCACCCCAACTTGTTTATTGCAGCTTATAATGGTTAC
AAATAAAGCAATAGCATCACAAAT
TTCACAAATAAAGCATTTTTTTCACTGCATTCTAGTTGTGGTTTGTCCAAACTCAT
CAATGTATCTTATCATGTCTGTAT
ACCGTCGACCTCTAGCTAGAGCTTGGCGTAATCATGGTCATAGCTGTTTCCTGTG
TGAAATTGTTATCCGCTCACAATTC
CACACAACATACGAGCCGGAAGCATAAAGTGTAAAGCCTGGGGTGCCTAATGAG
TGAGCTAACTCACATTAATTGCGTTG
CGCTCACTGCCCGCTTTCCAGTCGGGAAACCTGTCGTGCCAGCTGCATTAATGAA
TCGGCCAACGCGCGGGGAGAGGCGG
TTTGCGTATTGGGCGCTCTTCCGCTTCCTCGCTCACTGACTCGCTGCGCTCGGTCG
TTCGGCTGCGGCGAGCGGTATCAG
CTCACTCAAAGGCGGTAATACGGTTATCCACAGAATCAGGGGATAACGCAGGAA
AGAACATGTGAGCAAAAGGCCAGCAA

1. An expression vector for cloning the Class I viral fusion protein gene and as DNA vaccine candidate, the expression vector comprising:
i) a first transcription unit comprising a membrane protein gene (M protein gene) of Coronavirus, an envelope protein gene (E protein gene) of Coronavirus and an internal ribosome entry sites (IRES) sequence, wherein the IRES is inserted into the junction of the membrane protein gene and the envelop protein gene;
ii) a first eukaryotic promoter operably linked to the membrane protein gene wherein the first promoter is located upstream of the M protein gene and drives the expression of the first transcription unit;
iii) a second transcription unit comprising a SpikeCT gene of Coronavirus and a multiple cloning site (MCS) for cloning or in-framed insertion of Class I viral fusion protein gene, wherein the MCS is located at the beginning of SpikeCT gene and has restriction enzymes cutting sites; and
iv) a second eukaryotic promoter operably linked to the SpikeCT gene wherein the second promoter is located upstream of the SpikeCT gene and drives the expression of the second transcription unit;
wherein the transcription activity of the first eukaryotic promoter is stronger than the second eukaryotic promoter.

2. The expression vector of claim 1, wherein the Coronavirus is pig, human or avian coronvirus.

3. The expression vector of claim 1, wherein the Coronavirus pig TGEV coronvirus, human 229E coronvirus or human SARS virus.

4. The expression vector of claim 1, wherein the envelope protein gene has the sequence as set forth in SEQ ID NO: 1.

5. The expression vector of claim 1, wherein the membrane protein gene has the sequence as set forth in SEQ ID NO: 2.

6. The expression vector of claim 1, wherein the SpikeCT gene has the sequence as set forth in SEQ ID NO: 3.

7. The expression vector of claim 1, which further comprises a replication origin for the propagation plasmid in a host cell.

8. The expression vector of claim 1, which further comprises an antibiotics-resistant gene as the selection marker.

9. The expression vector of claim 1, wherein the first and second transcription units have polyA signal.

10. The expression vector of claim 9, wherein the polyA signal BGH polyA signal.

11. The expression vector of claim 1, which has the sequence as set forth in SEQ ID NO:49.

12. The expression vector of claim 1, wherein the SpikeCT gene encodes C-terminal heptad repeat located upstream of an aromatic residue-rich region juxtaposed to the transmembrane segment of Spike protein of coronavirus.

13. The expression vector of claim 1, the multiple cloning site comprises the restriction site selected from the group consisting of SmaI, BsaB I, EcoR V and BspE I.

14. The expression vector of claim 1, wherein the Class I viral fusion protein gene is selected from the group consisting of gp160 of HIV, HA of influenza virus and Spike of SARS virus.

15. The expression vector of claim 1, wherein the first eukaryotic promoter is selected from the group consisting of CMV, SV40, RSV, HIV-1 LTR, hybrid Beta-actin/CMV promoter enhancer, muscle-specific desmin, creatine kinase, beta-Actin promoter, EF1alpha, Ubiquitin promoter.

16. The expression vector of claim 1, wherein the first eukaryotic promoter is selected from the group consisting of pCMV, Hybrid Beta-actin/CMV promoter enhancer and beta-Actin promoter.

17. The expression vector of claim 1, wherein the second eukaryotic promoter is selected from the group consisting of CMV, SV40, RSV, HIV-1 LTR, hybrid Beta-actin/CMV promoter enhancer, muscle-specific desmin, creatine kinase, beta-Actin promoter, EF1alpha, Ubiquitin promoter.

18. The expression vector of claim 1, wherein the second eukaryotic promoter is selected from the group consisting of pCMV, SV40, beta-Actin and EF1alpha promoter.
